(19) United States
(12) Patent Application Publication
(10) Pub. No.: US 2024/0415494 A1
(43) Pub. Date: Dec. 19, 2024

(54) ULTRASONIC DIAGNOSTIC APPARATUS, DATA MANAGEMENT SYSTEM, DATA ESTIMATION METHOD, AND RECORDING MEDIUM

(71) Applicant: Konica Minolta, Inc., Tokyo (JP)

(72) Inventor: Yoshihiro TAKEDA, Tokyo (JP)

(21) Appl. No.: 18/678,435

(22) Filed: May 30, 2024

(30) Foreign Application Priority Data
Jun. 13, 2023 (JP) 2023-096663

(51) Int. Cl. A61B 8/08; A61B 8/00; G06T 7/00

(52) U.S. Cl. CPC A61B 8/5207; A61B 8/4444; A61B 8/463; A61B 8/5238; G06T 7/0012; G06T 2207/10132; G06T 2207/20081; G06T 2207/30004

(57) ABSTRACT

An ultrasonic diagnostic apparatus includes: a first ultrasonic probe that transmits and receives ultrasonic waves to and from a subject; and a hardware processor that uses a learned model machine-learned by using learning data including first data based on a reception signal for image generation received by a second ultrasonic probe of the same type as that of the first ultrasonic probe, and second data captured under a predetermined condition so as to convert third data based on a reception signal for image generation received by the first ultrasonic probe into fourth data similar to the second data, and output the converted data.

### CROSS REFERENCE TO RELATED APPLICATIONS

The present invention claims priority under 35 U.S.C. § 119 to Japanese Application No. 2023-096663, filed on Jun. 13, 2023, the entire contents of which being incorporated herein by reference.

### BACKGROUND OF THE INVENTION

### Technical Field

The present invention relates to an ultrasonic diagnostic apparatus, a data management system, a data estimation method, and a recording medium.

### Description of Related Art

Conventionally, an ultrasonic diagnostic apparatus has been known which irradiates the inside of a subject with ultrasonic waves by an ultrasonic probe, receives and analyzes its reflected waves, and thereby displays an ultrasonic image of the inside of the subject. The subject is a living body of a patient or the like.

In recent years, more ultrasonic diagnostic apparatuses have been used for various purposes by using artificial intelligence (AI) technology. Specifically, the ultrasonic diagnostic apparatus is applied to automatic discrimination (recognition) of a target organ/tissue from ultrasonic image data, and to automatic measurement by using the discrimination result.

For example, an ultrasonic image diagnostic apparatus is known which acquires a discrimination result outputted from a discriminator that discriminates a discrimination target object captured in an ultrasonic image (see JP 2022-172565 A). The discrimination target object is an organ, a body structure, a lesion, or an abnormal luminance region. The ultrasonic image diagnostic apparatus changes a discriminator to be used from a plurality of discriminators in accordance with a body part in which a user is interested, a measurement target, a processor that performs discrimination processing, and the type of an ultrasonic probe.

### SUMMARY OF THE INVENTION

A conventional ultrasonic diagnostic apparatus displays ultrasonic images with various image qualities in accordance with an ultrasonic probe, an apparatus, a manufacturer, and transmission/reception setting (frequency). However, in the case where the image is changed due to the above reason, an inexperienced user such as a new doctor or a paramedic staff might not know how to view the image, which might make it difficult to perform diagnosis. For the above reasons, it is said that an ultrasonic diagnostic apparatus is a modality highly dependent on the examination skill of a user. Variation in the examination skill has a high risk of leading to variation in diagnosis.

The ultrasonic image diagnostic apparatus described in JP 2022-172565 A displays ultrasonic images with various image qualities depending on the ultrasonic probe or the like and provides notification of a discrimination result of the ultrasonic image by AI. However, the user is required to understand the image content based on one ultrasonic image that is displayed, determine whether or not the discrimination result by AI is appropriate, and make a final diagnosis.

An object of the present invention is to enable even a user unexperienced in diagnosis to perform appropriate diagnosis from images with various image qualities.

To achieve at least one of the abovementioned objects, according to an aspect of the present invention, an ultrasonic diagnostic apparatus reflecting one aspect of the present invention includes:
a first ultrasonic probe that transmits and receives ultrasonic waves to and from a subject; and
a hardware processor that uses a learned model machine-learned by using learning data including first data based on a reception signal for image generation received by a second ultrasonic probe of the same type as that of the first ultrasonic probe, and second data captured under a predetermined condition so as to convert third data based on a reception signal for image generation received by the first ultrasonic probe into fourth data similar to the second data and output the converted data.

To achieve at least one of the abovementioned objects, according to an aspect of the present invention, a data estimation method reflecting one aspect of the present invention causes a hardware processor to use a learned model machine-learned by using learning data that includes: first data based on a reception signal for image generation received by a second ultrasonic probe of the same type as that of a first ultrasonic probe transmitting and receiving ultrasonic waves to and from a subject; and second data captured under a predetermined condition so as to convert third data based on a reception signal for image generation received by the first ultrasonic probe into fourth data similar to the second data, and output the converted data.

To achieve at least one of the abovementioned objects, according to an aspect of the present invention, a recording medium reflecting one aspect of the present invention is a non-transitory computer-readable recording medium storing a program that causes a computer to use a learned model machine-learned by using learning data including: first data based on a reception signal for image generation received by a second ultrasonic probe of the same type as that of a first ultrasonic probe transmitting and receiving ultrasonic waves to and from a subject; and second data captured under a predetermined condition so as to convert third data based on a reception signal for image generation received by the first ultrasonic probe into fourth data similar to the second data, and output the converted data.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating a schematic configuration of an ultrasonic diagnostic system according to an embodiment of the present invention,

FIG. 2 is a block diagram illustrating a functional configuration of an ultrasonic diagnostic apparatus,

FIG. 3 is a diagram illustrating a configuration of a GAN,

FIG. 4 is a diagram illustrating a configuration of a Cycle-GAN,

FIG. 5 is a flowchart illustrating first ultrasonic image output processing,

FIG. 6 is a diagram illustrating data and processing of the first ultrasonic image output processing,

FIG. 7 is a view showing a display screen,

FIG. 8A is a view showing an ultrasonic image,

FIG. 8B is a view showing an MRI-style image,

FIG. 9 is a flowchart illustrating second ultrasonic image output processing,

FIG. 10 is a flowchart illustrating third ultrasonic image output processing,

FIG. 11 is a diagram illustrating data and processing of the third ultrasonic image output processing; and

FIG. 12 is a flowchart illustrating fourth ultrasonic image output processing.

### DETAILED DESCRIPTION

Hereinafter, one or more embodiments of the present invention will be described with reference to the drawings. However, the scope of the invention is not limited to the disclosed embodiments. Hereinafter, a first embodiment, a modification example thereof, a second embodiment and a third embodiment of the present invention will be described in order in detail with reference to the accompanying drawings.

### First Embodiment

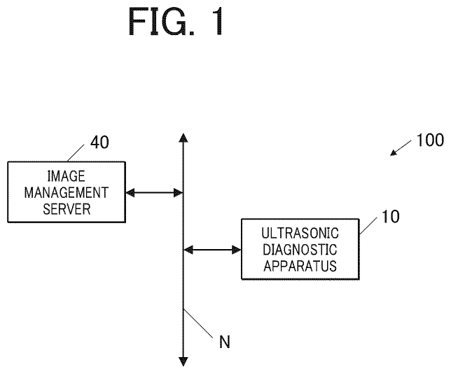

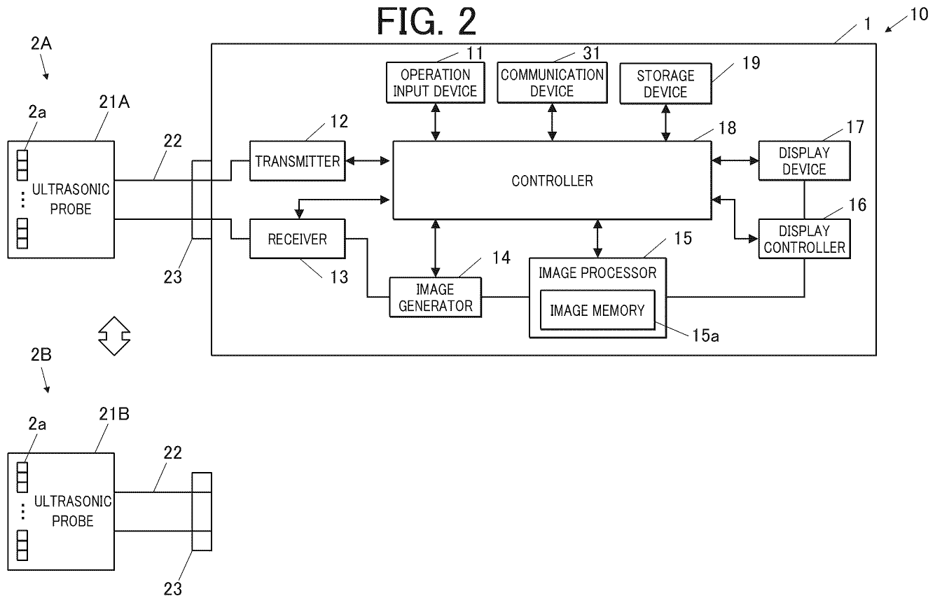

The first embodiment of the present invention will be described with reference to FIG. 1 to FIG. 8B. First, the apparatus configuration according to the present embodiment will be described with reference to FIG. 1 to FIG. 3. FIG. 1 illustrates a block diagram illustrating the schematic configuration of an image management system 100 according to the present embodiment. FIG. 2 is a block diagram illustrating the functional configuration of an ultrasonic diagnostic apparatus 10.

As illustrated in FIG. 1, the image management system 100 as a data management device system is installed in medical facility such as a hospital. The image management system 100 is a system that manages medical image data such as ultrasonic image data.

The image management system 100 includes an ultrasonic diagnostic apparatus 10, and an image management server 40 as a management device. The apparatuses of the image management system 100 are connected to each other via a communication network N so as to be able to perform mutual data communication. The communication network N is a local area network (LAN) or the like.

The image management server 40 is an apparatus of a picture archiving and communication system (PACS). The image management server 40 receives, stores, and manages ultrasonic image data generated by the ultrasonic diagnostic apparatus 10.

Note that it may be configured that an examination apparatus of a modality other than the ultrasonic diagnostic apparatus 10 is connected to the image management system 100. The examination apparatus is an MRI (magnetic resonance imaging apparatus), a CT (computed tomography apparatus), a DR (digital X-ray imaging apparatus), or the like. In this configuration, the image management server 40 manages medical image data generated by each of the above examination apparatuses.

As illustrated in FIG. 2, the ultrasonic diagnostic apparatus 10 includes an ultrasonic diagnostic apparatus body 1 and ultrasonic probes 2A, 2B. One of the ultrasonic probes 2A, 2B is connected to the ultrasonic diagnostic apparatus body 1. The ultrasonic probes 2A, 2B are different types of ultrasonic probes, respectively. The type of the ultrasonic probe is a model number, a product name, a version, or the like. However, the ultrasonic probes of the same type include not only ultrasonic probes having the same model number, the same product name, the same version, and the like, but also include physically the same ultrasonic probes (having the same manufacturing number). Here, the ultrasonic probe 2B has, for example, a relatively high frequency of ultrasonic waves and a function excellent in resolution of an ultrasonic image. The ultrasonic probe 2A has, for example, a relatively low frequency of ultrasonic waves and a function excellent in contrast of an ultrasonic image. Note that one or three or more types of ultrasonic probes may be connected to the ultrasonic diagnostic apparatus body 1.

The ultrasonic probes 2A, 2B transmit ultrasonic waves (transmission ultrasonic waves) to the inside of a subject and receive reflected waves (reflected ultrasonic waves: echoes) of the ultrasonic waves reflected inside the subject. The ultrasonic probe 2A includes an ultrasonic probe body 21A, a cable 22, and a connector 23. The ultrasonic probe 2B includes an ultrasonic probe body 21B, the cable 22, and a connector 23. The ultrasonic probe body 21A is a head part of the ultrasonic probe 2A and transmits and receives ultrasonic waves. The ultrasonic probe body 21B is a head part of the ultrasonic probe 2B and transmits and receives ultrasonic waves. The cable 22 is connected to the ultrasonic probe body 21A or 21B and the connector 23. The cable 22 is a cable through which drive signals and reception signals of the ultrasonic waves for the ultrasonic probe bodies 21A, 21B flow. The connector 23 is a connector of a plug for connecting to a connector (not illustrated) of a receptacle of the ultrasonic diagnostic apparatus body 1.

In addition, the connector 23 includes a storage section (not shown). This storage device stores discrimination information including the type of the ultrasonic probe of its own device. In a state where the ultrasonic probe is connected to the ultrasonic diagnostic apparatus body 1, the storage section of the connector 23 can be accessed from the ultrasonic diagnostic apparatus body 1 side.

The ultrasonic diagnostic apparatus body 1 is connected to the ultrasonic probe body 21A or 21B via the connector 23 and the cable 22. The ultrasonic diagnostic apparatus body 1 transmits a drive signal that is an electric signal to the ultrasonic probe body 21A or 21B. By transmitting the drive signal, the ultrasonic diagnostic apparatus body 1 causes the ultrasonic probe body 21A or 21B to transmit transmission ultrasonic waves to the subject. The ultrasonic probe 2A or 2B generates a reception signal that is an electric signal, in accordance with reflected ultrasonic waves reflected from the inside of the subject and received by the ultrasonic probe body 21A or 21B. Based on the reception signal generated by the ultrasonic probe 2A or 2B, the ultrasonic diagnostic apparatus body 1 images the internal state of the subject as ultrasonic image data.

The ultrasonic probe body 21A has transducers 2a on its tip end side. The transducers 2a include a plurality of transducers that are arranged, for example, in a one-dimensional array in an azimuth direction (scanning direction). Note that the transducers 2a may be arranged in a two-dimensional array. In addition, the number of transducers 2a can be set to any number. In the present embodiment, an electronic scanning probe of a linear scanning type is adopted as each of the ultrasonic probes 2A, 2B. However, the ultrasonic probes 2A, 2B may be of either an electronic scanning type or a mechanical scanning type. Further, the ultrasonic probes 2A, 2B may be of any one of a linear scanning type, a sector scanning type, and a convex scanning type. The ultrasonic diagnostic apparatus body 1 and the ultrasonic probe 2A or 2B may be configured to perform wireless communication instead of wired communication via the cable 22. The wireless communication uses an ultra wide band (UWB) or the like.

The ultrasonic diagnostic apparatus body 1 includes an operation input device 11, a transmitter 12, a receiver 13, an image generator 14, an image processor 15, a display controller 16, a display device 17, a controller 18, a storage device 19, and a communication device 31. The controller 18 functions as a first estimating section, a second estimating section, an estimation section, an output section, a switching unit, and a selection section.

The operation input device 11 includes operational elements such as a push button, an encoder, a lever switch, a joystick, a trackball, a keyboard, a touch pad, and a multifunction switch. The operation input device 11 receives an operation inputted to each of the operational elements from a user such as a doctor or a technician, and outputs the operation information to the controller 18.

Under the control of the controller 18, the transmitter 12 provides a drive signal that is an electric signal, to the ultrasonic probe 2A or 2B so as to cause the ultrasonic probe 2A or 2B to generate the transmission ultrasonic waves. The transmitter 12 includes, for example, a clock generation circuit, a delay circuit, and a pulse generation circuit. The clock generation circuit generates a clock signal that determines transmission timing and a transmission frequency of the drive signal. The delay circuit sets a delay time for each individual path corresponding to each transducer 2a and delays transmission of the drive signal by each set delay time. The delay circuit focuses a transmission beam composed of the transmission ultrasonic waves by the above delay. The pulse generation circuit generates a pulse signal as a drive signal at a predetermined cycle. The transmitter 12 generates the transmission ultrasonic waves by driving, for example, some in series (e.g., 64) of the plurality of (e.g., 192) transducers 2a arranged in the ultrasonic probe 2A or 2B. Then, the transmitter 12 performs scanning by shifting the transducer 2a that oscillates very time a transmission ultrasonic wave is generated in the azimuth direction (scanning direction).

The receiver 13 receives a reception signal that is an electric signal, from the ultrasonic probe 2A or 2B under the control of the controller 18. The receiver 13 includes, for example, an amplifier, an A/D conversion circuit, and a phasing addition circuit. The amplifier amplifies the reception signal by a preset amplification factor for each individual path corresponding to each of the transducers 2a. The A/D conversion circuit performs analog-to-digital conversion (A/D conversion) on the amplified reception signal. The phasing addition circuit provides a delay time to the A/D-converted reception signal for each individual path corresponding to each transducer 2a to adjust the time phase and adds (performs phasing addition on) these signals to generate sound ray data.

Under the control of the controller 18, the image generator 14 performs envelope detection processing, logarithmic compression, or the like on the sound ray data from the receiver 13 and performs brightness conversion on this data through adjustments of the dynamic range and gain of the data. Through the brightness conversion, the image generator 14 generates B (brightness)-mode image data including pixels having brightness values as received energy. That is, the B-mode image data represents an intensity of a reception signal by a brightness. The image generator 14 may be configured to generate tomographic image data in another image mode other than the B-mode, such as a color Doppler mode or an elasticity image mode. Furthermore, the image generator 14 may be configured to have another image mode for generating image data other than tomographic image data, such as a motion mode (M-mode).

The image processor 15 includes an image memory 15a. The image memory 15a includes, for example, a semi-conductor memory such as a dynamic random access memory (DRAM). Under the control of the controller 18, the image processor 15 performs appropriate image processing on the B-mode image data inputted from the image generator 14. Under the control of the controller 18, the image processor 15 stores the B-mode image data, which is inputted or image-processed, in the image memory 15a on a frame-by-frame basis. The B-mode image data per frame unit may be referred to as ultrasonic image data. Under the control of the controller 18, the image processor 15 transmits the ultrasonic image data stored in the image memory 15a to the display controller 16 frame by frame at predetermined time intervals. The image memory 15a is composed of, for example, a large-capacity memory capable of holding frame image data for about 10 seconds. The image memory 15a holds ultrasonic image data for the latest 10 seconds by a first-in first-out (FIFO) method.

Under the control of the controller 18, the display controller 16 performs processing of coordinate conversion or the like on the B-mode image data inputted from the image processor 15 to convert this data into an image signal for display. The display controller 16 outputs the image signal to the display device 17.

The display device 17 includes a display panel of a liquid crystal display (LCD), an organic electroluminescence (OEL) display, an inorganic electroluminescent (IEL) display, or the like. Under the control of the controller 18, the display device 17 displays an ultrasonic image on the display panel in accordance with the image signal outputted from the display controller 16. Furthermore, the display device 17 displays various display information inputted from the controller 18 on the display panel.

The controller 18 includes, for example, a central processing unit (CPU), a read only memory (ROM), and a random access memory (RAM). The controller 18 reads out various processing programs stored in the ROM, develops them in the RAM, and controls each component of the ultrasonic diagnostic apparatus 10 in cooperation with the developed programs and the CPU. The ROM is composed of a nonvolatile memory or the like, such as a semiconductor. The ROM stores a system program corresponding to the ultrasonic diagnostic apparatus 10, various processing programs executable on the system program, various data or the like, such as a gamma table. These programs are stored in the RAM in form of computer-readable program codes. The CPU sequentially executes operation in accordance with the program codes on the RAM. The RAM forms a work area where various programs executed by the CPU and data related to these programs are temporarily stored.

In particular, the ROM of the controller 18 stores a first ultrasonic image output program for executing first ultrasonic image output processing described later.

The storage device 19 is a storage device such as a hard disk drive (HDD) or a solid state drive (SSD), which stores information regarding ultrasonic image data or the like in a writable and readable manner. In particular, the storage device 19 stores a converter and a discriminator as learned models of AI described later.

The communication device 31 is connected to the communication network N. The communication device 31 includes a network card, and others. The controller 18 transmits and receives information to and from an external device such as the image management server 40 connected to the communication network N, via the communication device 31.

Regarding each component included in the ultrasonic diagnostic apparatus 10, some or all of the functions of the respective functional blocks can be implemented as hardware circuits such as integrated circuits. The integrated circuit is, for example, a large scale integration (LSI). An LSI may be referred to as an integrated circuit (IC), a system LSI, a super LSI, or an ultra LSI depending on the degree of its integration. Further, the method of circuit integration is not limited to an LSI. The circuit integration may be realized by a dedicated circuit or a general-purpose processor, or by using a field programmable gate array (FPGA) or a reconfigurable processor in which the connection and setting of circuit cells in an LSI can be reconfigured. In addition, each component may execute a part or all of the functions of the functional blocks by software. In this case, the software is stored in one or more storage media such as ROMs, optical discs, or hard disks. An arithmetic processor of the ultrasonic diagnostic apparatus 10 reads out the software from the storage media and executes the software.

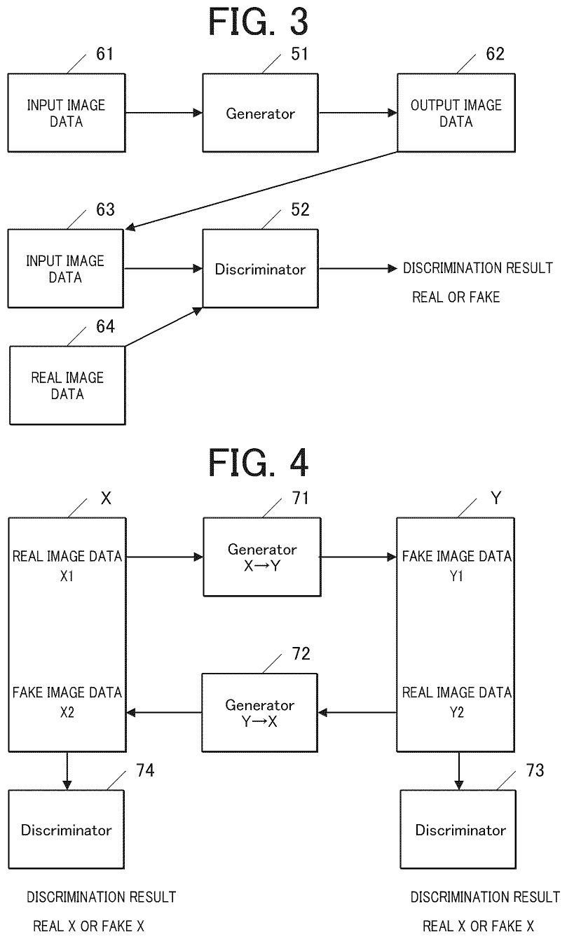

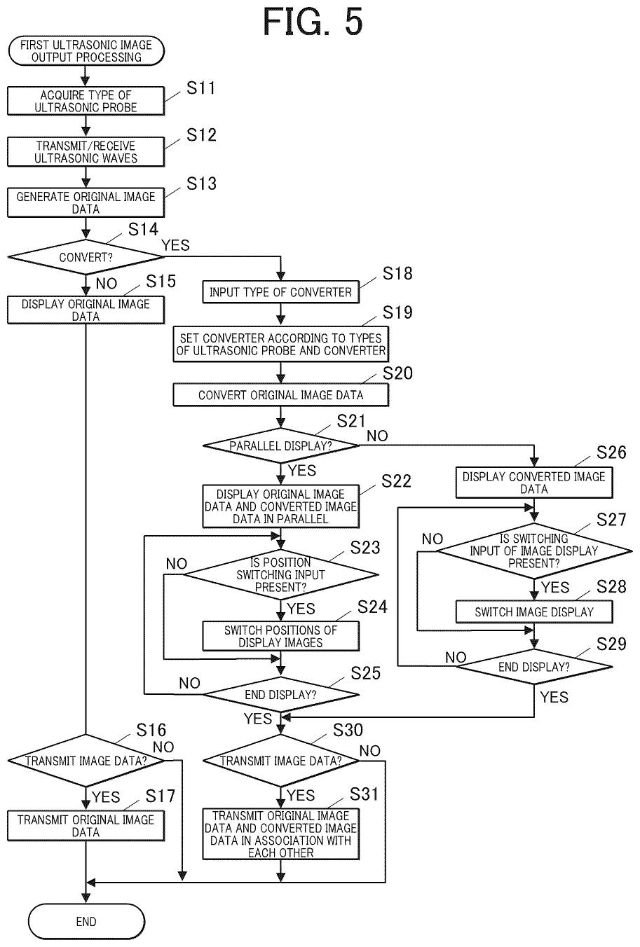

Next, an AI algorithm used for image data conversion according to the present embodiment will be described with reference to FIG. 3 and FIG. 4. Here, as the AI algorithm, a generative adversarial network (GAN) and a Cycle-GAN will be described. FIG. 3 is a diagram illustrating the configuration of the GAN. FIG. 4 is a diagram illustrating the configuration of the Cycle-GAN.

The GAN is a type of AI algorithm used in unsupervised learning that requires no labeling of data. By learning (machine-learning) features from data, the GAN can generate non-existent data and convert the data in accordance with features of existent data.

The GAN is implemented by a system of two neural networks that compete with each other in a zero-sum game framework. Two neural networks are a generator network (generator) and a discriminator network (discriminator). As in the present embodiment, if the purpose is to generate image data, a generator outputs image data. A discriminator discriminates (determines) whether or not the image data outputted from the generator is correct. The generator learns to deceive the discriminator. The discriminator learns to execute more accurate discrimination. As described above, the two networks learn on the basis of contrary purposes, and this is the reason why they are called “adversarial”.

As illustrated in FIG. 3, the GAN includes a generator 51 and a discriminator 52. The generator 51 converts input image data 61 and outputs output image data 62. The generator 51 performs the conversion such that the output image data 62 is discriminated as real image data by the discriminator 52.

On the other hand, in the discriminator 52, the output image data 62 is set as input image data 63, and this input image data 63 and real image data 64 are inputted to the discriminator 52. The discriminator 52 discriminates whether the input image data 63 and the real image data 64 are real image data or fake image data, and outputs a discrimination result. At this time, the discriminator 52 learns to discriminate the input image data 63 as fake image data and the real image data 64 as real image data. Using the discrimination result of the discriminator 52, the generator 51 learns such that the output image data 62 is discriminated as real image data by the discriminator 52. The above learning is repeated.

For example, it is assumed that the initial input image data 61 is image data having random pixel values. It is assumed that the real image data 64 is B-mode image data. The output image data 62 at the initial stage of learning is image data of a messed-up image. As the learning is repeated, the output image data 62 gradually becomes an image looking more real. The output image data 62 at the end of learning becomes an image looking much more real like the real image data 64.

In the present embodiment, a Cycle-GAN which is a type of a GAN is used. The Cycle-GAN is a GAN that extracts a predetermined feature of an image of one image data group X, and converts image data of the image data group X into image data with a predetermined feature of the other image data group Y. The features in the image data groups X, Y include, for example, a pair of (horse and zebra) or a pair of (summer scenery and winter scenery), or the like. Further, the Cycle-GAN can mutually convert image data between the image data group X and the image data group Y.

As shown in FIG. 4, the Cycle-GAN includes generators 71, 72, and discriminators 73, 74. The generator 71 converts real image data X1 having a feature of the image data group X and outputs the converted image data as fake image data Y1 having a feature of the image data group Y. The generator 71 performs the conversion such that the fake image data Y1 is discriminated as real image data of the image data group Y by the discriminator 73.

On the other hand, the fake image data Y1 and real image data Y2 having the feature of the image data group Y are inputted to the discriminator 73. The discriminator 73 discriminates whether the fake image data Y1 and the real image data Y2 are real image data or fake image data of the image data group Y, and outputs a discrimination result. At this time, the discriminator 73 learns to discriminate the fake image data Y1 as fake image data and the real image data Y2 as real image data of the image data group Y. Using the discrimination result of the discriminator 73, the generator 71 learns such that the fake image data Y1 is discriminated as real image data by the discriminator 73.

The generator 72 converts the real image data Y2 of the image data group Y and outputs this data as the fake image data Y1 having the feature of the image data group X. The generator 72 performs conversion such that the fake image data X2 is discriminated as the real image data of the image data group X by the discriminator 74.

In the meantime, the fake image data X2 and the real image data X1 are inputted to the discriminator 74. The discriminator 74 discriminates whether the fake image data X2 and the real image data X1 are real image data or fake image data of the image data group X, and outputs a discrimination result. At this time, the discriminator 74 learns to discriminate the fake image data X2 as fake image data of the image data group X, and discriminate the real image data X1 as real image data. Using the discrimination result of the discriminator 74, the generator 72 learns such that the fake image data X2 is discriminated as real image data by the discriminator 74.

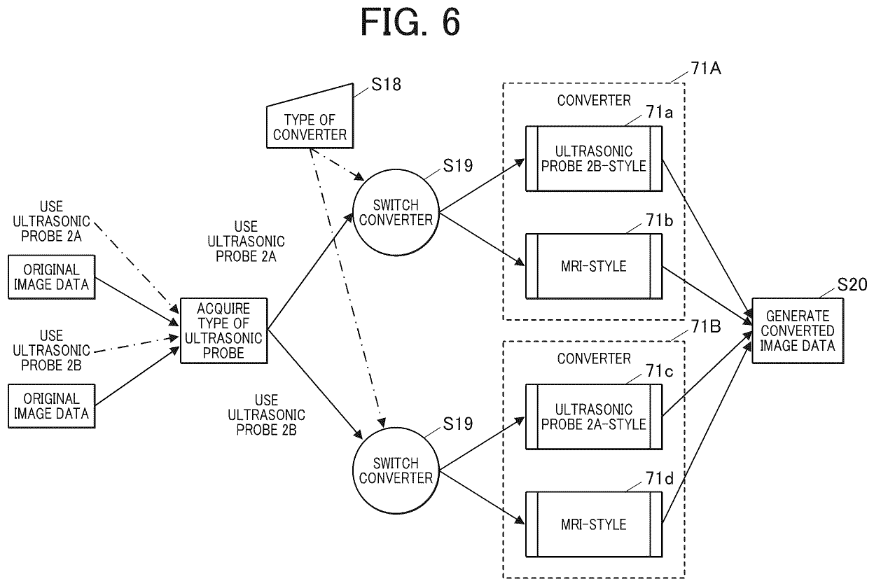

In the present embodiment, the storage device 19 stores the generator 71. The pairs of features (attributes) of the image data groups X, Y include: (the ultrasonic probe 2A, the ultrasonic probe 2B); (the ultrasonic probe 2A, an MRI); and (the ultrasonic probe 2B, an MRI). Specifically, the storage device 19 stores the generators 71A, 71B (FIG. 6) as the generator 71. The generator 71A is a converter for image data generated by using the ultrasonic probe 2A. The generator 71B is a converter for image data generated by using the ultrasonic probe 2B. The generator 71A includes generators 71a, 71b. The generator 71B includes generators 71c, 71d.

The “(ultrasonic) image data generated by using the ultrasonic probe” means “(ultrasonic) image data based on a reception signal for image generation received by the ultrasonic probe”. Hereinafter, the “(ultrasonic) image data generated by using the ultrasonic probe” is simply referred to as “(ultrasonic) image data using the ultrasonic probe”.

The generator 71a performs image conversion to convert the image quality of the ultrasonic image data using the ultrasonic probe 2A into an image quality looking like the ultrasonic image using the ultrasonic probe 2B (ultrasonic probe 2B-style). The generator 71b performs image conversion to convert the image quality of the ultrasonic image data using the ultrasonic probe 2A into an image quality looking like an MRI image (MRI-style). The generator 71c performs image conversion to convert the image quality of the ultrasonic image data using the ultrasonic probe 2B into an image quality looking like the ultrasonic image using the ultrasonic probe 2A (ultrasonic probe 2A-style). The generator 71d performs image conversion to convert the image quality of the ultrasonic image data using the ultrasonic probe 2B into an image quality in the MRI-style.

The generators 71a, 71b have been learned using the ultrasonic image data using the ultrasonic probe 2A and the ultrasonic image data using the ultrasonic probe 2B. The discriminators 73, 74 corresponding to the generators 71a, 71c have also been learned in the same manner. For example, the real image data X1 is ultrasonic image data using the ultrasonic probe 2A. The real image data Y2 is ultrasonic image data using the ultrasonic probe 2B.

The generator 71b and the discriminators 73, 74 corresponding to this generator have been learned by using the ultrasonic image data using the ultrasonic probe 2A and the MRI image data. The generator 71d and the discriminators 73, 74 corresponding to this generator have been learned by using the ultrasonic image data using the ultrasonic probe 2B and the MRI image data.

The Cycle-GAN can advance the learning without using strictly paired learning data (training data). Specifically, the real image data of the image data group X as training data and the real image data of the image data group Y may be different from each other in the cross section or positioning of a subject.

In the case of using the generator 71 for the image conversion from X to Y, essentially, the real image data of the image data group X as the training data may be any image data. However, in order to increase the conversion accuracy, it is preferable that the real image data of the image data group X as the training data is limited to one feature (e.g., the ultrasonic probe 2A) before the conversion. In this manner, the Cycle-GAN relatively facilitates collection of the learning data. Furthermore, the Cycle-GAN can convert the image data to image data having a feature of a target desired to be converted.

As the learning progresses, the converted image data converted by the generator 71 becomes image data looking more similar to the real image data Y2. For example, the fake image data Y1 becomes image data looking similar to the real image data Y2. “Image data looking similar” is defined as image data having the same image feature in the Cycle-GAN, and having similar contrasts, gradations, graininess, and sharpness between the images. “Image data looking similar to the real image data Y2” is defined, for example, as image data (e.g., tomographic image data) having the same imaging direction as that of the real image data Y2.

The real image data Y2 is image data imaged under predetermined conditions. The image data imaged under the predetermined conditions is, for example, image data using an ultrasonic probe of a different type from that of the ultrasonic probe used for the real image data X1.

Hereinafter, the generators 71, 71A, 71B, and 71a to 71d are referred to as converters 71, 71A, 71B, 71a to 71d. The discriminators 73, 74 are referred to as discriminators 73, 74.

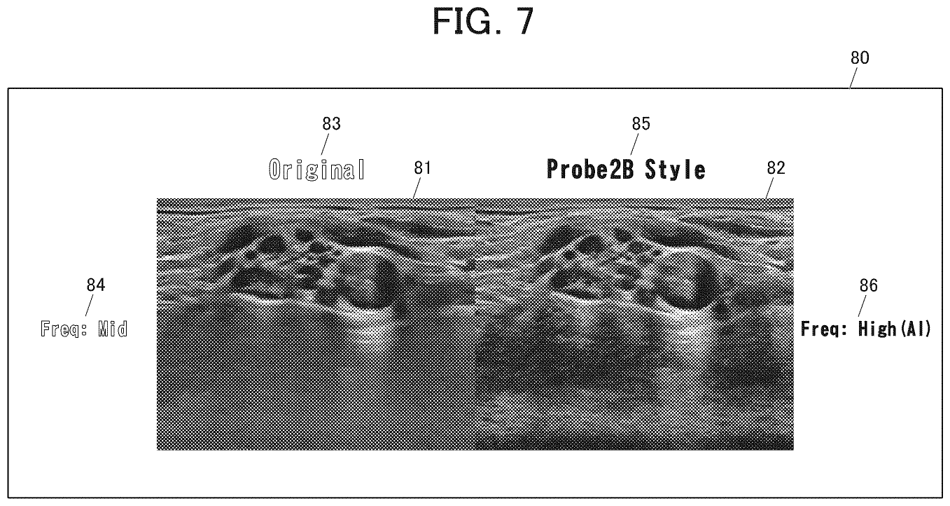

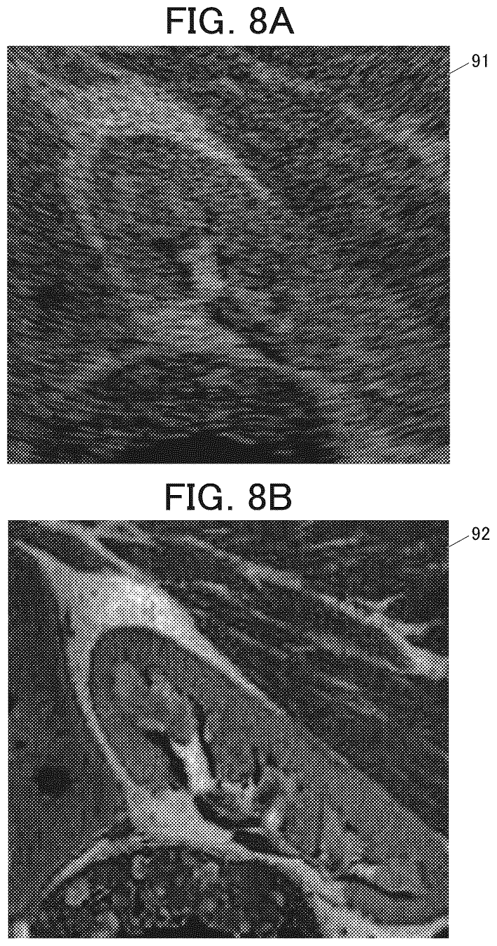

Next, with reference to FIG. 5 to FIG. 8B, the operation of the image management system 100 will be described. FIG. 5 is a flowchart illustrating the first ultrasonic image output processing. FIG. 6 is a diagram illustrating data and processing of the first ultrasonic image output processing. FIG. 7 is a view showing a display screen 80. FIG. 8A is a view showing an ultrasonic image 91. FIG. 8B is a view showing an MRI-style image 92.

With reference to FIG. 5, the first ultrasonic image output processing executed by the ultrasonic diagnostic apparatus 10 will be described. It is assumed that the ultrasonic probe 2A or 2B is connected to the ultrasonic diagnostic apparatus body 1 in advance. It is also assumed that the converters 71a to 71d are stored in the storage device 19.

In the ultrasonic diagnostic apparatus 10, for example, an execution instruction to execute the first ultrasonic image output processing is inputted from a user via the operation input device 11. In response to the input of the execution instruction, the controller 18 executes the first ultrasonic image output processing in accordance with the first ultrasonic image output program stored in the ROM.

First, the controller 18 acquires the type of the ultrasonic probe connected to the ultrasonic diagnostic apparatus body 1 (step S11). The controller 18 reads out discrimination information of the ultrasonic probe connected to ultrasonic diagnostic apparatus body 1, the discrimination information being stored in the storage section of the connector 23 of this ultrasonic probe. The controller 18 acquires the type of the ultrasonic probe corresponding to the discrimination information read out. As illustrated in FIG. 6, the type of the ultrasonic probe is, for example, the “ultrasonic probe 2A” or the “ultrasonic probe 2B”.

The controller 18 controls the transmitter 12 and the receiver 13 to transmit and receive ultrasonic waves via the ultrasonic probe (step S12). In step S12, the user pushes a tip end of the ultrasonic probe against a subject so as to perform scanning.

The controller 18 controls the receiver 13, the image generator 14, the image processor 15, and the display controller 16 to generate ultrasonic image data based on the ultrasonic waves in step S12 (step S13). The ultrasonic image data in step S13 is B-mode image data and is defined as original image data. As illustrated in FIG. 6, for example, original image data using the ultrasonic probe 2A or original image data using the ultrasonic probe 2B is generated.

The controller 18 receives, from the user via the operation input device 11, an input of setting information regarding whether or not to convert the original image data by the discriminator of AI (step S14). In step S14, based on the setting information inputted, the controller 18 determines whether or not to convert the original image data.

If determining not to convert the original image data (NO in step S14), the controller 18 causes the display device 17 to display the original image data generated in step S13 (step S15). The controller 18 receives, from the user via the operation input device 11, an input regarding whether or not to transmit the image data to the image management server 40 (step S16). In step S16, in response to the input, the controller 18 determines whether or not to transmit the image data to the image management server 40.

If it is determined not to transmit the image data (NO in step S16), the first ultrasonic image output processing is ended. If determining to transmit the image data (YES in step S16), the controller 18 transmits the original image data to the image management server 40 via the communication device 31 (step S17). In response to step S17, the image management server 40 receives the original image data from the ultrasonic diagnostic apparatus 10 and stores this original image data in the storage device of its own server. After step S17, the first ultrasonic image output processing is ended.

If determining to convert the original image data (YES in step S14), the controller 18 receives an input regarding the type of the converter of AI from the user via the operation input device 11 (step S18). As illustrated in FIG. 6, the type of the converter of AI is, for example, the converter 71a, 71b, 71c, or 71d. In step S18, it is sufficient that the feature of the converter after the conversion (e.g., the ultrasonic probe 2A-style, the ultrasonic probe 2B-style, or the MRI-style) may be at least inputted.

The controller 18 sets the converter to be used in accordance with the type of the ultrasonic probe acquired in step S11 and the type of the converter inputted in step S18 (step S19). As shown in FIG. 6, for example, one of the converters 71a, 71b, 71c, and 71d is set as the converter to be used. Here, as the converter to be used, a converter is set which has the same type of the ultrasonic probe acquired in step S11 and the same type of the ultrasonic probe of the real image data X1 in FIG. 4, and also corresponds to the type of the converter in step S18.

The controller 18 reads out the converter set in step S19 from the storage device 19 and converts the original image data in step S13 by this converter (step S20). The converted original image data is defined as converted image data. As shown in FIG. 8, for example, when the converter 71a is used, the converted image data becomes, for example, processed image data in the ultrasonic probe 2B-style having an image quality with the feature of an ultrasonic image using the ultrasonic probe 2B. When the converter 71b or 71d is used, the converted image data becomes processed image data in the MRI-style having an image quality with the feature of an MRI image. Furthermore, for example, when the converter 71c is used, the converted image data becomes processed image data in the ultrasonic probe 2A-style having an image quality with the feature of an ultrasonic image using the ultrasonic probe 2A.

The controller 18 receives, from the user via the operation input device 11, an input regarding whether or not to display the image data in parallel (step S21). In step S21, the controller 18 determines whether or not to display the image data in parallel in accordance with the input information.

If determining to perform the parallel display (YES in step S21), the controller 18 displays the original image data in step S13 and the converted image data in step S20 in parallel on the display device 17 (step S22). In step S22, for example, a display screen 80 illustrated in FIG. 7 is displayed. The display screen 80 corresponds to the case where the original image data is generated by using the ultrasonic probe 2A and is subjected to image conversion by using the converter 71a.

The display screen 80 includes an original image 81, a converted image 82, and supplementary information 83, 84, 85, 86. The original image 81 is an image of the original image data using the ultrasonic probe 2A. The converted image 82 is an image of the converted image data obtained by converting the original image data of the original image 81 by using the converter 71a. The original image 81 and the converted image 82 are arranged in parallel in the right-left direction. Note that the original image 81 and the converted image 82 may be arranged side by side in the up-down direction.

The supplementary information 83 is information indicating that the original image 81 is an original image. The supplementary information 83 is, for example, text information showing “Original” arranged near the original image 81. However, the supplementary information 83 may be other text information, a mark, a frame of the original image 81, or the like. The supplementary information 84 is supplementary information regarding the original image 81. The supplementary information 83 is, for example, text information showing “Freq: Mid” arranged near the original image 81. “Freq: Mid” indicates that the frequencies for transmission and reception of the ultrasonic waves of the original image 81 are set at a middle level. The supplementary information 83, 84 is expressed in white, for example.

The supplementary information 85 is information indicating that the converted image 82 is a processed image of the original image, and also indicating the content thereof. The supplementary information 85 is, for example, text information showing “Probe 2B Style” arranged near the original image 81. “Probe 2B Style” indicates that the converted image 82 is a converted image having an image quality with the feature of the “ultrasonic probe 2B-style”. However, the supplementary information 83 may be other text information, a mark, a frame of the converted image 82, or the like. The supplementary information 86 is supplementary information related to the converted image 82. The supplementary information 86 is, for example, text information showing “Freq: High (AI)” arranged near the original image 81. “Freq: High (AI)” indicates that the frequencies for transmission and reception of the ultrasonic waves of the converted image 82 are set at a high level and indicates that this image is a processed image processed by AI. The supplementary information 85, 86 is expressed in black, for example. As described above, the supplementary information 83, 84 and the supplementary information 85, 86 are set in different colors from each other, thereby discriminatively display the original image and the converted image (processed image).

Returning to FIG. 5, the controller 18 receives a switching input to switch the positions of these two images being displayed, from the user via the operation input device 11 (step S23). In step S23, in response to the input, the controller 18 determines whether or not there is a switching input to switch the positions of these two images being displayed. If there is the switching input (YES in step S23), the controller 18 switches the positions between the original image and the converted image that are displayed in parallel (step S24). In step S24, for example, while the display screen 80 is being displayed, the display is switched to the parallel display in which the converted image 82 is arranged on the right side and the original image 81 is arranged on the left side.

The controller 18 receives an input to end the display of the images, from the user via the operation input device 11 (step S25). In step S25, in response to the input, the controller 18 determines whether or not to end the display of the images. If there is no switching input (NO in step S23), the process shifts to step S25. If it is determined not to end the display (NO in step S25), the process shifts to step S23.

If determining not to perform the parallel display (NO in step S21), the controller 18 displays the converted image data in step S20 alone on the display device 17 (step S26). In step S26, for example, on the display screen 80, only the converted image 82 and the supplementary information 85, 86 are displayed.

The controller 18 receives a switching input to switch the display of one image being displayed, from the user via the operation input device 11 (step S27). In step S27, in response to the input, the controller 18 determines whether or not there is a switching input to switch the display of the image being displayed. If there is the switching input (YES in step S27), the controller 18 switches the display from the image being displayed to the other image corresponding to this image (step S28). In step S28, for example, when only the converted image 82 and the supplementary information 85, 86 are displayed, the display is switched to a display showing only the original image 81 and the supplementary information 83, 84.

The controller 18 receives an input to end the display of the image, from the user via the operation input device 11 (step S29). In step S29, in response to the input, the controller 18 determines whether or not to end the display of the image. If it is determined not to end the display (NO in step S29), the process shifts to step S27.

If determining to end the display (YES in step S29), the controller 18 receives an input regarding whether or not to transmit the image data, from the user via the operation input device 11 (step S30). If it is determined to end the display (YES in step S29), the process shifts to step S30. In step S30, in response to the input, the controller 18 determines whether or not to transmit the image data to the image management server 40.

If it is determined not to transmit the image data (NO in step S30), the first ultrasonic image output processing is ended. If determining to transmit the image data (YES in step S30), the controller 18 associates the original image data and the converted image data with each other and transmits this associated image data to the image management server 40 via the communication device 31 (step S31). Corresponding to step S31, the image management server 40 receives the original image data and the converted image data from the ultrasonic diagnostic apparatus 10. The image management server 40 stores the original image data and the converted image data in association with each other in the storage device of its own server. After step S31, the first ultrasonic image output processing is ended.

The display screen 80 is an example in which the converter 71a is used to convert the original image data of the original image 81 using the ultrasonic probe 2A into the converted image data of the converted image 82 in the ultrasonic probe 2B-style. As shown in FIG. 8A, for example, original image data of an ultrasonic image 91 that is an ultrasonic image using the ultrasonic probe 2A is considered. As shown in FIG. 8B, the converter 71b is used to convert the original image data of the ultrasonic image 91 into converted image data of an MRI-style image 92 having an image quality with an MRI feature.

As described above, according to the present embodiment, the ultrasonic diagnostic apparatus 10 includes: a first ultrasonic probe as the ultrasonic probe 2A or 2B that transmits and receives ultrasonic waves to and from the subject; and the controller 18 as the estimation unit. The controller 18 uses the converter 71 as a learned model that is machine-learned using learning data. The learning data includes: first data based on a reception signal for image generation received by a second ultrasonic probe of the same type as that of the first ultrasonic probe; and second data imaged under a predetermined condition. The first data is the real image data X1 in FIG. 4. The second data is the real image data Y2 in FIG. 4. The controller 18 converts third data based on the reception signal for image generation received by the first ultrasonic probe into fourth data similar to the second data, and outputs this fourth data. The third data is the original image data using the first ultrasonic probe. The fourth data is converted image data similar to the real image data Y2.

Therefore, even a user unexperienced in diagnosis can perform appropriate diagnosis from the images (the original image and the converted image) having various image qualities, using the original image data and the converted image data.

The first data (real image data X1) and the third data (original image data) are B-mode image data based on a predetermined reception signal. Therefore, even a user unexperienced in diagnosis can perform appropriate diagnosis from the images (the original image and the converted image) having various image qualities, using the original image data and the converted image data.

The second data (real image data Y2) includes ultrasonic image data and MRI image data. Therefore, it is possible to perform appropriate diagnosis from the images (the ultrasonic image, the MRI-style image) having image qualities with various features.

The fourth data (converted image data) is ultrasonic image-style image data or MRI-style image data. Therefore, it is possible to perform appropriate diagnosis from the images (the ultrasonic image, the MRI-style image) having image qualities with various features.

The first data (real image data X1) is data based on the reception signal for image generation received by the second ultrasonic probe. The second data (real image data Y2) is data based on the reception signal for image generation received by the third ultrasonic probe as the ultrasonic probe 2A or 2B, which is different from the second ultrasonic probe. Therefore, by using the image data in the third ultrasonic probe-style, it is possible to perform appropriate diagnosis from the images (the ultrasonic image of the first ultrasonic probe and the third ultrasonic probe-style image) having various image qualities.

The controller 18 displays the supplementary information 85, 86 indicating that the fourth data (converted image data) is a processed image, on the display device 17. Therefore, by notifying the user of the fourth data being a processed image, thereby alerting the user that the fourth data that is the processed image is unsuitable for definitive diagnosis and or final confirmation. In addition, it is possible to prevent confusion between the original image and the converted image that are displayed.

The controller 18 displays the supplementary information 85, 86 indicating the type and frequency of the ultrasonic probe corresponding to the fourth data (converted image data), on the display device 17. When the fourth data (converted image data) is MRI-style image data, the supplementary information may include the type of modality (MRI) instead of the type of the ultrasonic probe. Therefore, it is possible to notify the user of the type, the frequency, and the modality of the ultrasonic probe corresponding to the fourth data, and it is possible to notify the user that the fourth data is subjected to inference processing.

The controller 18 displays the supplementary information 83, 84 indicating that the third data (original image data) is an original image, on the display device 17. Therefore, it is possible to notify (clearly indicate) the user that the third data is suitable for definitive diagnosis and or final confirmation and is an original image. In addition, it is possible to prevent confusion between the displayed original image and the converted image (processed image).

The controller 18 displays the third data (original image data), or displays the third data and the fourth data (converted image data) in association with each other. Accordingly, it is possible to prevent the user from performing definitive diagnosis and or final confirmation with the processed image, and to prompt the user to perform definitive diagnosis and or final confirmation with the original image.

The image management system 100 includes the ultrasonic diagnostic apparatus 10 and the image management server 40. The image management server 40 receives from the ultrasonic diagnostic apparatus 10, and stores the third data (original image data) or the third data and the fourth data (converted image data) in association with each other. The controller 18 transmits the third data (original image data) or the third data and the fourth data (converted image data) in association with each other to the image management server 40. Accordingly, the image management server 40 can receive the third data alone, or the third data and the fourth data, and store and manage these data in association with each other. Note that the controller 18 may output the third data (original image data) or the third data and the fourth data (converted image data) in association with each other, on a radiology report.

The third data is the original image data. In addition, the fourth data is the converted image data. The ultrasonic diagnostic apparatus 10 includes the display device 17 that displays the original image data and the converted image data, or switchingly displays either one of them, on the same screen. Accordingly, the converted image and the original image can be displayed side by side with each other or can be switchingly displayed, which allows the user to choose a preferable display method, thereby performing appropriate diagnosis.

### Modification Example

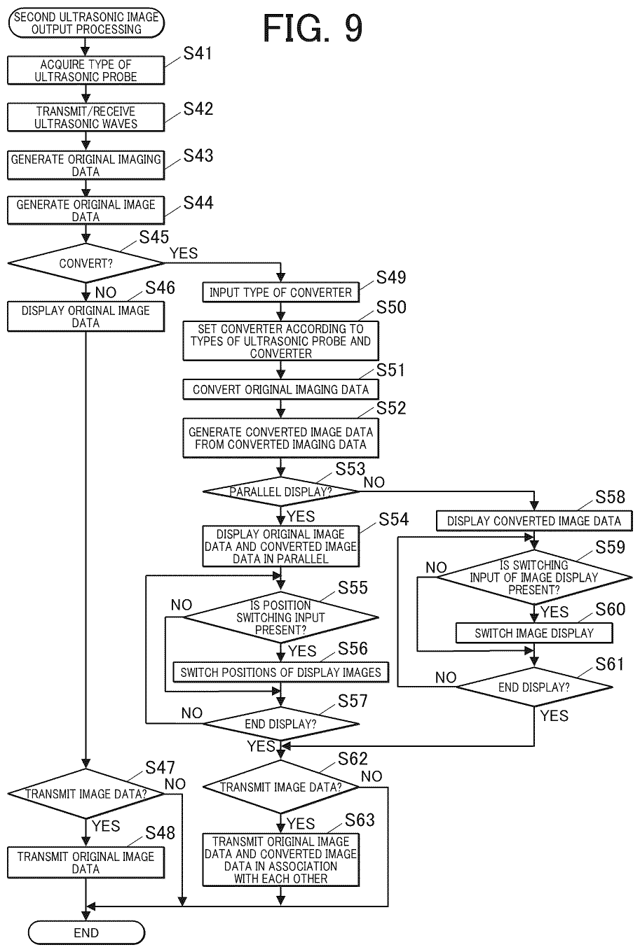

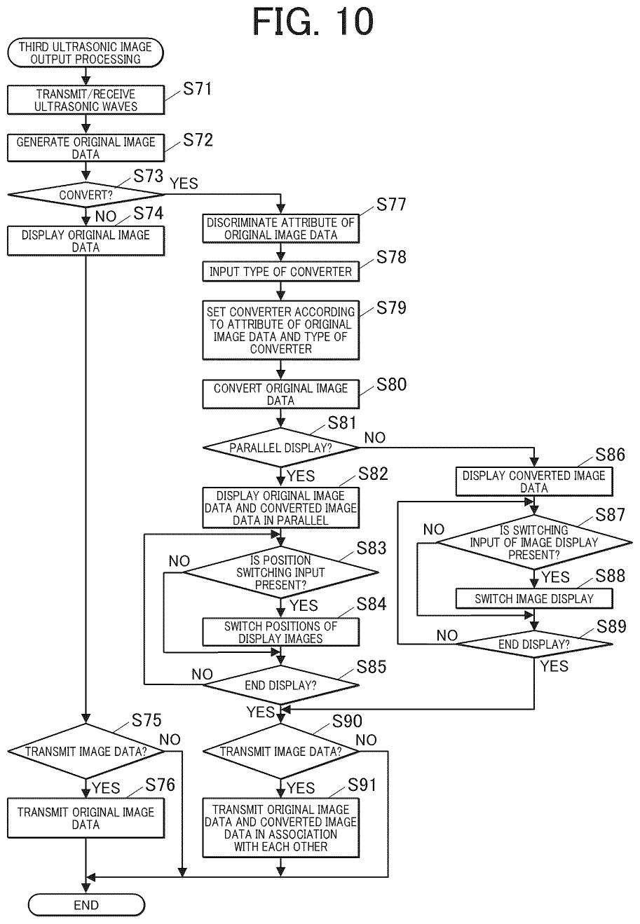

A modification example of the first embodiment will be described with reference to FIG. 9. FIG. 9 is a flowchart illustrating second ultrasonic image output processing.

The above first embodiment is configured to convert the original image data into the converted image data. The present modification example is configured to convert sound ray data before imaging of an original image into sound ray data before imaging of a converted image. The sound ray data before imaging of the original image is defined as original imaging data. The sound ray data before imaging of the converted image is defined as converted imaging data.

As the apparatus configuration of the present modification example, the image management system 100 is adopted as in the above first embodiment. Note that instead of the first ultrasonic image output program, a second ultrasonic image output program is stored in the ROM of the controller 18 of the ultrasonic diagnostic apparatus 10. The second ultrasonic image output program is a program to execute second ultrasonic image output processing described later.

Next, the operation of the image management system 100 will be described with reference to FIG. 9. Specifically, with reference to FIG. 9, the second ultrasonic image output processing executed by the ultrasonic diagnostic apparatus 10 will be described. In the ultrasonic diagnostic apparatus 10, it is assumed that the ultrasonic probe 2A or 2B is connected to the ultrasonic diagnostic apparatus body 1 in advance. It is also assumed that the converters 71a to 71d are stored in the storage device 19.

In the ultrasonic diagnostic apparatus 10, for example, an execution instruction to execute the second ultrasonic image output processing is inputted from the user via the operation input device 11. In response to the input of the execution instruction, the controller 18 executes the second ultrasonic image output processing in accordance with the second ultrasonic image output program stored in the ROM.

Step S41 and step S42 shown in FIG. 9 are the same as step S11 and step S12 of the first ultrasonic image output processing shown in FIG. 5. The controller 18 controls the receiver 13 to generate sound ray data of an ultrasonic image based on the ultrasonic waves in step S12 (step S43). The sound ray data in step S43 is imaging data for generating a B-mode image and is defined as original imaging data. The controller 18 controls the image generator 14, the image processor 15, and the display controller 16 to generate an ultrasonic image data based on the original imaging data in step S43 (step S44). The ultrasonic image data in step S44 is B-mode image data and is defined as original image data.

The controller 18 receives, from the user via the operation input device 11, an input of setting information regarding whether or not to convert the original imaging data by the discriminator of AI (step S45). In step S45, based on the setting information inputted, the controller 18 determines whether or not to convert the original imaging data.

If determining not to convert the original imaging data (NO in step S45), the controller 18 causes the display device 17 to display the original image data generated in step S44 (step S46). Step S47 to step S50 are the same as step S16 to step S19 in FIG. 5. The controller 18 reads out the converter set in step S50 from the storage device 19 and converts the original imaging data in step S43 by this converter (step S51). The original imaging data that is converted is defined as converted imaging data.

The controller 18 controls the image generator 14, the image processor 15, and the display controller 16 to generate converted image data from the converted imaging data in step S51 (step S52). Step S53 to step S63 are the same as step S21 to step S31 in FIG. 5.

As described above, according to the present modification example, the first data (the sound ray data corresponding to the real image data X1) and the third data (the sound ray data corresponding to the original image data) are the sound ray data as a predetermined reception signal. Therefore, as in the first embodiment, even a user unexperienced in diagnosis can perform appropriate diagnosis from the images (the original image and the converted image) having various image qualities by using the original image data and the converted image data.

The second data (real image data Y2) includes ultrasonic data (sound ray data) and MRI image data. Therefore, it is possible to perform appropriate diagnosis from the images (the ultrasonic image and the MRI-style image) having image qualities with various features.

The fourth data (converted image data) is ultrasonic data (sound ray data) and MRI-style image data. Therefore, it is possible to perform appropriate diagnosis from the images (the ultrasonic image and the MRI-style image) having image qualities with various features.

As described in the first embodiment or the present modification example, the first data and the third data are in the same data format (the image data or the sound ray data). For example, when the first data is B-mode image data, it is preferable that the third data is also B-mode image data. When the first data is sound ray data, it is preferable that the third data is also sound ray data. In the case of this configuration, the conversion accuracy of the converter can be increased.

Although the present modification example is configured to use the sound ray data as the reception signals of the first data and the third data, the reception signal is not limited to this. A radio frequency (RF) signal of the receiver 13 may be used as the reception signal.

### Second Embodiment

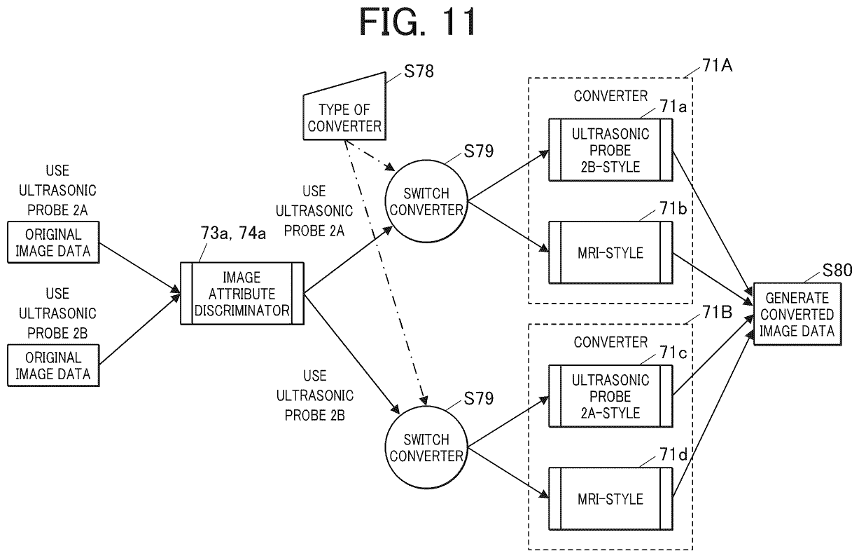

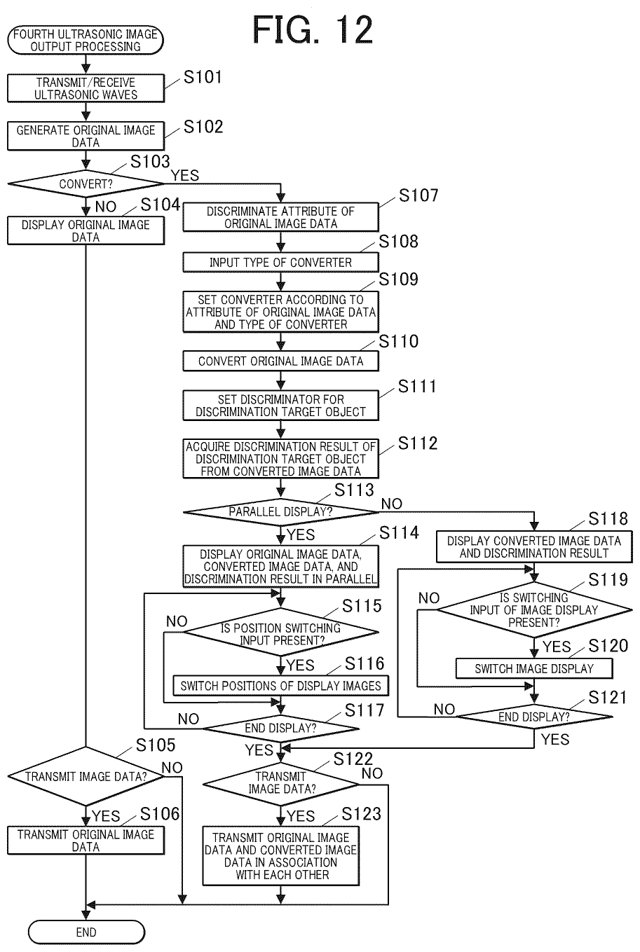

A modification example of the second embodiment of the present invention will be described with reference to FIG. 10 and FIG. 11. FIG. 10 is a flowchart illustrating third ultrasonic image output processing. FIG. 11 is a diagram illustrating data and processing of the third ultrasonic image output processing.

The above first embodiment is configured to read out, from the storage device, the type of the ultrasonic probe connected to the ultrasonic diagnostic apparatus body 1 as the attribute of the original image data and use this type of the ultrasonic probe for setting the converter. The present embodiment is configured to discriminate the type of the ultrasonic probe as the attribute of the original image data by using a discriminator of AI and use this discriminated type of the ultrasonic probe for setting the converter.

The apparatus configuration of the present embodiment uses the image management system 100, as in the first embodiment. However, instead of the first ultrasonic image output program, a third ultrasonic image output program is stored in the ROM of the controller 18 of the ultrasonic diagnostic apparatus 10. The third ultrasonic image output program is a program to execute the third ultrasonic image output processing described later.

Next, the operation of the image management system 100 will be described with reference to FIG. 10 and FIG. 11. Specifically, the third ultrasonic image output processing executed by the ultrasonic diagnostic apparatus 10 will be described with reference to FIG. 10. It is assumed that in the ultrasonic diagnostic apparatus 10, the ultrasonic probe 2A or 2B is connected to the ultrasonic diagnostic apparatus body 1 in advance. In addition, as illustrated in FIG. 11, it is assumed that the converters 71a to 71d and the discriminators 73a, 74b as the discriminators 73, 74 are stored in the storage device 19.

The discriminator 73a is a discriminator that discriminates the ultrasonic image data using the ultrasonic probe 2B from the real image data. The discriminator 74a is a discriminator that discriminates the ultrasonic image data using the ultrasonic probe 2A from the real image data. The discriminators 73a, 74a have been learned, for example, by using the ultrasonic image data using the ultrasonic probe 2A and the ultrasonic image data using the ultrasonic probe 2B in FIG. 4. For example, the real image data X1 is ultrasonic image data using the ultrasonic probe 2A. The real image data Y2 is ultrasonic image data using the ultrasonic probe 2B.

In the ultrasonic diagnostic apparatus 10, for example, an execution instruction to execute the third ultrasonic image output processing is inputted, for example, from the user via the operation input device 11. In response to the input of the execution instruction, the controller 18 executes the third ultrasonic image output processing in accordance with the third ultrasonic image output program stored in the ROM.

Step S71 to step S76 illustrated in FIG. 9 are the same as step S12 to step S17 of the first ultrasonic image output processing in FIG. 5. If determining to convert the original image data (YES in step S73), the controller 18 reads out the discriminators 73a, 73b from the storage device 19 (step S77). In step S77, the controller 18 discriminates the image attribute of the original image data by using the discriminators 73a, 73b.

For example, when an original image data is inputted to the discriminator 73a and a discrimination result showing that the image data is real is obtained, the “ultrasonic probe 2B” is obtained as the attribute of the original image data. When an original image data is inputted to the discriminator 74a and a discrimination result showing that the image data is real is obtained, the “ultrasonic probe 2B” is obtained as the attribute of the original image data. That is, as the attribute of the original image data, the type of the ultrasonic probe is obtained.

Step S78 corresponds to step S18 in FIG. 5. The controller 18 sets a converter to be used in accordance with the attribute of the original image data acquired in step S77 and the type of the converter inputted in step S78 (step S79). Step S80 to step S91 are the same as step S20 to step S31 in FIG. 5.

As described above, according to the present embodiment, the learned models stored in the storage device 19 include a plurality of learned models including at least a first learned model and a second learned model. The first learned model (the converter 71) has been machine-learned by using the learned data including first data and second data. The second learned model (the discriminators 73a, 74a) has been machine-learned by using the learned data including fifth data and sixth data. The fifth data is based on the reception signal for image generation received by the second ultrasonic probe of the same type as that of the first ultrasonic probe. The sixth data indicates an attribute of the fifth data. The controller 18 performs switching to an output of any one of the plurality of learned models by operation of a user. The controller 18 selects the first learned model in accordance with the attribute of the third data (original image data) discriminated by the second learned model and with the operation of the user. The controller 18 converts the third data into the fourth data similar to the second data by using the first learned model selected, and outputs this fourth data. Accordingly, the attribute of the third data can be easily acquired from the third data by using the second learned model.

The controller 18 includes a plurality of switching sections (“SWITCH CONVERTER” in FIG. 11) corresponding to each attribute of the third data. The controller 18 discriminates the attribute of the third data and selects one of the plurality of switching sections. Accordingly, the attribute of the third data can be easily acquired from the third data by using the second learned model.

### Third Embodiment

With reference to FIG. 12, a modification example of the third embodiment of the present invention will be described. FIG. 12 is a flowchart illustrating fourth ultrasonic image output processing.

The above first embodiment is configured to convert the original image data to generate, display, and transmit the converted image data. The present embodiment is configured to discriminate a discrimination target object such as a predetermined organ/tissue in an image from the converted image data.

The apparatus configuration of the present embodiment uses the image management system 100, as in the first embodiment. However, instead of the first ultrasonic image output program, a fourth ultrasonic image output program is stored in the ROM of the controller 18 of the ultrasonic diagnostic apparatus 10. The fourth ultrasonic image output program is a program to execute the fourth ultrasonic image output processing described later.

Next, the operation of the image management system 100 will be described with reference to FIG. 12. Specifically, the fourth ultrasonic image output processing executed by the ultrasonic diagnostic apparatus 10 will be described with reference to FIG. 12. In the ultrasonic diagnostic apparatus 10, it is assumed that the ultrasonic probe 2A or 2B is connected to the ultrasonic diagnostic apparatus body 1 in advance. It is also assumed that a discriminator D1 (not illustrated) for discriminating the discrimination target object of the subject is stored in the storage device 19.

The discriminator D1 is a discriminator of AI that discriminates the predetermined discrimination target object from an image of converted image data converted from original image data by the converter 71. The discriminator D1 is, for example, a learned model composed by a convolutional neural network. The discrimination target object is at least one of an organ and a tissue. An organ is a heart, a liver, or the like. A tissue is an organ, a nerve, fascia, a muscle, a blood vessel, a placenta, a lymph node brain, a prostate, a carotid artery, a breast, or the like. In addition, the discrimination target object may be a lesion portion indicating some disease, an abnormal luminance region in an ultrasonic image of interest, or the like.

The discriminator D1 is learned by using the converted image data including the discrimination target object in the image and a predetermined correct label of this converted image data. The predetermined correct label includes an imaging region, an imaging portion, a position, measurement, image classification of the discrimination target object, and others. When the converted image data converted from the original image data by the converter 71 is inputted to the discriminator D1, the discriminator D1 discriminates the discrimination target object in the image of the converted image data, and outputs the discrimination result. For example, the discriminator D1 is prepared for each type of the discrimination target object.

The discriminator D1 is conventionally learned by using image data and a correct label for each ultrasonic probe. In this case, for example, when a lesion portion is a discrimination target object, it is difficult to obtain a discriminator having sufficient performance unless learning data is prepared for each corresponding probe. In order to add a new ultrasonic probe, it is necessary to newly prepare learning data for this new probe; therefore, it takes time to create a discriminator. Furthermore, in order to cope with a plurality of ultrasonic probes, each learned model is required to cope with images having various image qualities. In such a case, since the size of each learned model increases, the real-time performance required for the ultrasonic diagnostic apparatus is deteriorated.

To the contrary, if the converted image data is used as an input, it is possible to reduce variation in image quality, which should be supported by the discriminator. This means that each learned model can be reduced in size. In addition, when a corresponding ultrasonic probe is newly added, it is necessary to create only the converter 71, thereby greatly reducing learning data for the discriminator. Here, an image including a lesion portion is not essential for the learning data used for creating the converter 71; therefore, sufficient learning data can be easily prepared and created.

In the ultrasonic diagnostic apparatus 10, an execution instruction to execute the fourth ultrasonic image output processing is inputted, for example, from the user via the operation input device 11. In response to the input of the execution instruction, the controller 18 executes the fourth ultrasonic image output processing in accordance with the fourth ultrasonic image output program stored in the ROM.

As illustrated in FIG. 12, step S101 to step S110 are the same as step S71 to step S80 of the third ultrasonic image output processing in FIG. 10. The controller 18 receives an input of the type of the discrimination target object desired to be discriminated, from the user via the operation input device 11 (step S111). In step S111, the controller 18 reads out the discriminator D1 corresponding to the inputted type of the discrimination target object, from the storage device 19, and sets this discriminator as the discrimination.

The controller 18 uses the discriminator set in step S111 to discriminate the discrimination target object in the converted image data in step S110 and acquire the discrimination result (step S112). The processing in step S113 is the same as the processing in step S81 in FIG. 10. If determining to perform parallel display (YES in step S113), the controller 18 displays the original image data in step S13 and the converted image data in step S20 in parallel on the display device 17 (step S114). In step S114, the controller 18 displays the discrimination result acquired in step S112 together with the original image data and the converted image data. The discrimination result is a discrimination result of the discrimination target object in the converted image, and is displayed near the converted image, for example.

Each processing of step S115 to step S117 is the same as each processing of step S83 to step S85 in FIG. 10. If determining not to perform the parallel display (NO in step S113), the controller 18 causes the display device 17 to display the converted image data in step S110 alone (step S118). In step S118, the controller 18 displays the discrimination result acquired in step S112 together with the converted image data. Step S119 to step S123 are the same as step S87 to step S91 in FIG. 10.

As described above, according to the present embodiment, the controller 18 uses the discriminator D1 to output the fifth data (discrimination result) from the fourth data (converted image data). The discriminator D1 has been machine-learned by using the learning data in which the fourth data obtained by converting the first data using the learned model (converter 71), and a predetermined correct label are made into a data set. Therefore, the discrimination target object such as an organ/tissue can be easily discriminated from the fourth data.

In the above description, as the computer-readable media of the programs according to the present invention, an example of using a ROM has been disclosed, but the present invention is not limited to this example. As other computer-readable media, a nonvolatile memory such as a flash memory and a portable recording medium such as a CD-ROM can be applied. Furthermore, as media for providing data of the programs according to the present invention via a communication line, carrier waves are also applicable to the present invention.

Note that the descriptions in the above embodiments are of examples of the ultrasonic diagnostic apparatus, the image management system, the data estimation method, and the recording medium according to the present invention, and the present invention is not limited to them. For example, at least two of the above embodiments and modification example may be combined as appropriate.

Further, in the above embodiments, the features of the second data (real image data Y2) are two types of the ultrasonic probes 2A, 2B, which are different from the ultrasonic probe used for the first data (real image data X1). However, the present invention is not limited to this. The features of the second data and the fourth data corresponding to the second data may be one or three or more types of ultrasonic probes.

Furthermore, in the above embodiments, the feature of the second data (real image data Y2) is the MRI that is a modality different from the ultrasonic diagnostic apparatus used for the third data (original image data). However, the present invention is not limited to this. The feature of the second data may be another modality such as CT.

Furthermore, the feature of the second data (real image data Y2) may be an ultrasonic diagnostic apparatus of a different type from that of the ultrasonic diagnostic apparatus used for the first data (real image data X1). The ultrasonic diagnostic apparatus used for the third data (original image data) is of the same type as that of the ultrasonic diagnostic apparatus used for the first data. The feature of the fourth data (converted image data) corresponds to the ultrasonic diagnostic apparatus having the feature of the second data. In this configuration, the supplementary information 83 to 86 in FIG. 11 and the like include information regarding the model name and the like of the ultrasonic diagnostic apparatus corresponding to the original image and the converted image.

In addition, the detailed configuration and the detailed operation of the image management system 100 according to the above embodiments can be appropriately changed without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus comprising:
a first ultrasonic probe that transmits and receives ultrasonic waves to and from a subject; and
a hardware processor that uses a learned model machine-learned by using learning data including first data based on a reception signal for image generation received by a second ultrasonic probe of the same type as that of the first ultrasonic probe, and second data captured under a predetermined condition,
so as to convert third data based on a reception signal for image generation received by the first ultrasonic probe into fourth data similar to the second data and output the converted data.

2. The ultrasonic diagnostic apparatus according to claim 1, wherein the first data and the third data are B-mode image data based on a predetermined reception signal or the reception signals.

3. The ultrasonic diagnostic apparatus according to claim 1, wherein the second data includes at least one of ultrasonic data, MRI data, and CT data.

4. The ultrasonic diagnostic apparatus according to claim 1, wherein the fourth data is ultrasonic data, MRI-style image data, or CT-style image data.

5. The ultrasonic diagnostic apparatus according to claim 1, wherein
the first data is data based on the reception signal for image generation received by the second ultrasonic probe, and
the second data is data based on a reception signal for image generation received by a third ultrasonic probe of a different type from that of the second ultrasonic probe.

6. The ultrasonic diagnostic apparatus according to claim 1, wherein the hardware processor outputs information indicating that the fourth data is a processed image.

7. The ultrasonic diagnostic apparatus according to claim 1, wherein the hardware processor outputs at least one of information regarding a type of an ultrasonic probe corresponding to the fourth data, a type of modality thereof, and a frequency thereof.

8. The ultrasonic diagnostic apparatus according to claim 1, wherein the hardware processor outputs information indicating that the third data is an original image.

9. The ultrasonic diagnostic apparatus according to claim 1, wherein the hardware processor outputs the third data, or outputs the third data and the fourth data in association with each other.

10. The ultrasonic diagnostic apparatus according to claim 1, wherein
the third data is third image data and the fourth data is fourth image data, and
the apparatus further comprises a display device that displays or switchingly displays the third image data and the fourth image data on a same screen.

11. The ultrasonic diagnostic apparatus according to claim 1, wherein
the learned model includes a plurality of learned models including: at least
a first learned model machine-learned by using learning data including the first data and the second data; and
a second learned model machine-learned by using learning data including fifth data based on the reception signal for image generation received by the second ultrasonic probe of the same type as that of the first ultrasonic probe, and sixth data indicating an attribute of the fifth data,
the hardware processor performs switching to an output of one learned model of the plurality of learned models by operation of a user, and
the hardware processor converts the third data into the fourth data similar to the second data by using the first learned model selected according to a switching unit and an attribute of the third data discriminated by the second learned model, and outputs the converted data.

12. The ultrasonic diagnostic apparatus according to claim 11, wherein
the hardware processor switches a plurality of switching sections, and
the hardware processor discriminates the third data and selects one switching section from the plurality of switching sections.

13. The ultrasonic diagnostic apparatus according to claim 1, wherein the hardware processor outputs fifth data from the fourth data by using a discriminator that is machine-learned by using learning data in which the fourth data obtained by converting the first data using the learned model, and a predetermined correct label are made into a data set.

14. A data management system comprising:
the ultrasonic diagnostic apparatus according to claim 1; and
a management device that receives the third data, or the third data and the fourth data from the ultrasonic diagnostic apparatus and stores the received data.

15. A data estimation method using a learned model machine-learned by using learning data that includes:
first data based on a reception signal for image generation received by a second ultrasonic probe of the same type as that of a first ultrasonic probe transmitting and receiving ultrasonic waves to and from a subject; and
second data captured under a predetermined condition
so as to convert third data based on a reception signal for image generation received by the first ultrasonic probe into fourth data similar to the second data and output the converted data.

16. A non-transitory computer-readable recording medium storing a program that causes a computer to use a learned model machine-learned by using learning data including:
first data based on a reception signal for image generation received by a second ultrasonic probe of the same type as that of a first ultrasonic probe transmitting and receiving ultrasonic waves to and from a subject; and
second data captured under a predetermined condition
so as to convert third data based on a reception signal for image generation received by the first ultrasonic probe into fourth data similar to the second data and output the converted data.
